(19) United States
(12) Patent Application Publication
(10) Pub. No.: US 2006/0067904 A1
(43) Pub. Date: Mar. 30, 2006

(54) Cosmetic compositions containing amphiphilic silicone resin emulsifier

(76) Inventors: Julio Gans Russ, Westfield, NJ (US); Jean Marie Manelski, Spring Lake, NJ (US); Tatyana Zamyatin, Princeton Junction, NJ (US); Ida Marie Sandewicz, Monroe Township, NJ (US); Merry Lee Nickl, Kingston, NJ (US); Nykol Annette West, Plainfield, NJ (US); Travis Duong, Toms River, NJ (US)

Correspondence Address: Julie Blackburn;Revlon Consumer Products Corporation, 237 Park Avenue, New York, NY, 10017, US

(21) Appl. No.: 10/955,050

(22) Filed: Sep. 30, 2004

(51) Int. Cl. A61K 8/89

(52) U.S. Cl. 424/70.12

(57) ABSTRACT

An internally compatible cosmetic composition comprising at least two internally incompatible phases or ingredients that are compatibilized with an effective amount of an amphiphilic silicone resin emulsifier and a method for emulsifying an anhydrous or emulsion cosmetic composition containing at least two internally incompatible phases with an amphiphilic silicone resin emulsifier either alone or in combination with a secondary emulsifier.

### TECHNICAL FIELD

The invention is in the field of cosmetic compositions for application to keratinous surfaces such as skin, hair, or nails for the purposes of conditioning, beautifying, coloring, or otherwise cosmetically treating the keratinous surfaces.

### BACKGROUND OF THE INVENTION

Many cosmetic compositions are found in the form of emulsions, either water-in-oil or oil-in-water. For example, foundation makeup, concealers, creams and lotions, and the like are typically found in the emulsion form. Emulsions are a very desirable vehicle for applying color or other actives to skin. The presence of water provides a light feeling evaporable film on the skin when the composition is applied, which dries to an aesthetically pleasing finish. However, one problem with compositions in the emulsion form is that they are difficult to formulate. In order to have products that are commercially acceptable, it is necessary that the emulsions exhibit a certain degree of stability in various tests. For example, cosmetics are often shipped under conditions where they are exposed to temperatures that are higher and lower than standard room temperature (72° F.). Products must be stable at these temperature extremes so that they can be delivered to the customer in a form that is suitable for commercial sale. In addition, commercially acceptable cosmetics must also be shelf stable, such that if they are stored on store shelves and not moved for long periods of time, they do not exhibit an inordinate degree of separation.

Thus, emulsifiers or surfactants are important in formulating cosmetic products that exhibit characteristics required for commercially acceptable products. The term “emulsifier” and “surfactant” are very similar, if not identical, in meaning. Both terms refer to ingredients that typically have amphiphilic properties. The term “amphiphilic” means that the ingredient has both lipophilic and hydrophilic portions such that the lipophilic portion is compatible with the lipophilic, or fatty, ingredients in the formula and the hydrophilic portion is compatible with the hydrophilic ingredients in the formula. The amphiphilic ingredient facilitates compatibility between two phases that may not otherwise be compatible to form a composition that is internally compatible. In the case where the composition has two internally incompatible phases that are not miscible, such as oil and water, the amphiphilic ingredient will, either alone or in combination with other emulsifiers, facilitate the dispersion of one phase into the other phase to create an oil and water emulsion that does not readily phase separate into water and oil phases upon standing. In the case where compositions are anhydrous, emulsifiers are often desirable to permit polar ingredients such as iron oxides, or hydrophilic ingredients such as mono-, di-, or polyhydric alcohols to be adequately dispersed in a lipophilic phase. For example, dispersing iron oxide pigments in a fatty phase is very often facilitated by using emulsifiers because certain iron oxide pigments may have charged, or polar, surfaces.

Typical emulsifiers for both anhydrous and emulsion products include silicone emulsifiers or organic emulsifiers. While these types of emulsifiers are widely available, there are certain drawbacks to their use. While the very popular silicone surfactants such as dimethicone copolyol or cetyl dimethicone copolyol are inexpensive and effective, they can be difficult to formulate with, and compositions containing them are sometimes prone to premature separation. In addition, when used in high concentrations they can be somewhat irritating for consumers having overly sensitive skin. Accordingly, there is a need for cosmetic compositions that are stable and compatible, and containing emulsifiers that do not have these drawbacks, and which are particularly suitable for use with silicone-based systems.

It is an object of the invention to provide cosmetic compositions having at least two internally incompatible phases that are compatibilized, in whole or in part, using an amphiphilic silicone resin emulsifier.

It is a further object of the invention to provide cosmetic compositions having at least two internally incompatible phases that are compatibilized using an amphiphilic silicone resin emulsifier and at least one other organic or silicone surfactant.

It is a further object of the invention to provide color cosmetic compositions in the water and oil emulsion form containing an amphiphilic silicone resin emulsifier.

It is a further object of the invention to provide anhydrous cosmetic compositions containing an amphiphilic silicone resin emulsifier.

### SUMMARY OF THE INVENTION

The invention comprises an internally compatible cosmetic composition comprising at least two internally incompatible phases that are compatibilized with an effective amount of an amphiphilic silicone resin emulsifier.

The invention further comprises a color cosmetic composition in the water and oil emulsion form containing an amphiphilic silicone resin emulsifier.

The invention further comprises an anhydrous color cosmetic composition containing an amphiphilic silicone resin emulsifier.

The invention further comprises a method for emulsifying an anhydrous or emulsion cosmetic composition containing at least two internally incompatible phases with an amphiphilic silicone resin emulsifier either alone or in combination with a secondary emulsifier.

### DETAILED DESCRIPTION

I. Definitions

A. All percentages mentioned herein are percentages by weight unless otherwise indicated.

B. The term “emulsifier” and “surfactant” are used interchangeably herein, and mean an ingredient with amphiphilic properties that has at least one hydrophilic portion and at least one lipophilic portion such that the ingredient is capable of improving the compatibility of two different phases or ingredients that have lipophilic or hydrophilic properties or moieties respectively.

C. The term “phase” or “phases” means one ingredient alone or a combination of ingredients that exhibit an affinity for each other. For example, in the cosmetic composition of the invention prior to inclusion of the amphiphilic silicone resin emulsifier, a phase in the formula that contains one ingredient may be incompatible with another formula phase containing another ingredient; or a phase containing one ingredient may be incompatible with another phase containing a combination of ingredients; or a combination of ingredients in the formula forming a phase may be incompatible with another combination of ingredients in the formula. One example is where a polar iron oxide pigment may be incompatible with an oil in the composition such that the iron oxide pigment will not readily solubilize or disperse in the oil. Another example is where combination of ingredients such as water and glycols may be incompatible with a silicone oil forming the oily phase of the composition. Another example is combination of ingredients such as water and glycols that form a phase, that may be incompatible with a combination of lipophilic oils in the emulsion.

D. The term “internally incompatible” means, with respect to two phases, where the two phases do not readily solubilize or disperse in each other. One non-limiting example of two internally incompatible phases may be found in a water and oil emulsion where the water and oil phases are not generally miscible, or where they may be miscible upon shaking the composition, but will readily settle out into separate water and oil phases upon standing. Another non-limiting example of two internally incompatible ingredients may be found in an anhydrous composition comprising an oil, or lipophilic phase, having dispersed therein polar iron oxide pigments as the colorant. In general, examples of internally incompatible phases are found where the formula includes ingredients or phases that are polar and nonpolar, or in the case where two incompatible ingredients are found in the same phase (such as the oily phase), where the solubility parameters of the two ingredients are different enough so that they are compatible with each other even though they are the same type (e.g. fatty) of material.

E. The term “compatibilize” or “improve compatibility” means that the two phases become more compatible with each other such that the two phases or ingredients are more readily miscible, soluble, or dispersible in, or with, each other when the amphiphilic silicone resin emulsifier is added to the composition.

F. The term “amphiphilic” means an ingredient or molecule that has hydrophilic portions and lipophilic portions, and exhibits compatibility with phases or ingredients that have hydrophilic or lipophilic properties or moieties.

G. The term “resin” with respect to the amphiphilic silicone resin, means that the ingredient is crosslinked and contains T units either alone or in combination with D units (as further defined below), and which may contain M or Q units.

II. The Composition

The compositions of the invention preferably comprise at least two phases that are internally incompatible, and which may be compatibilized, in whole or in part, with at least one amphiphilic silicone resin emulsifier either alone or in combination with one or more additional emulsifiers.

A. The Amphiphilic Silicone Resin Emulsifier

The amphiphilic silicone resin emulsifier may be present in the composition ranging from about 0.001-85%, preferably about 0.005-75%, more preferably about 0.01-65% by weight of the total composition. The amphiphilic silicone resin emulsifier may comprise T and/or D units, either alone or in combination with M and Q units. The resin comprises predominantly T and/or D units, and may comprise minor amounts of M or Q units due to the manner in which such resins are typically made. The amphiphilic silicone resin emulsifier must contain sufficient hydroxyl and/or alkoxy or similar hydrophilic or lipophilic functional groups to provide an ingredient that provides amphiphilic properties. Generally, the amphiphilic silicone resin emulsifiers used in the compositions of the invention generally contain from about ______ to ______ mole weight percent of polar (such as hydroxyl) functionality in order to provide the desired amphiphilic properties with the lipophilic property generally being found in the silxoane backbone whether substituted or unsubstituted. In particular, when substituted with methyl groups a lipophilic property is provided.

The term “trifunctional siloxy unit” is generally designated by the letter “T” in standard silicone nomenclature. A “T” unit has the general formula:
R₁ SiO_{3/2}
wherein R₁ is each independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆); hydroxyl; or hydrogen. The SiO_{3/2} designation means that the silicon atom is bonded to three oxygen atoms when the unit is copolymerized with one or more of the other units. For example when R₁ is methyl the resulting trifunctional unit is of the formula:

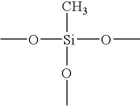

When this trifunctional unit is polymerized with one or more of the other units, the silicon atom shares three oxygen atoms with other silicon atoms, i.e. will share three halves of an oxygen atom.

The term “difunctional siloxy unit” is generally designated by the letter “D” in standard silicone nomenclature. If the D unit is substituted with substituents other than methyl the “D′” designation is sometimes used, which indicates a substituent other than methyl. For purposes of this disclosure, a “D” unit has the general formula:
R₁R₂SiO_{2/2}
wherein R₁ and R₂ are as defined above with respect to R₁. Preferred is where in the D unit, R₁ is methyl and R₂ is hydroxyl. The SiO_{2/2} designation means that the silicon atom in the difunctional unit is bonded to two oxygen atoms when the unit is polymerized with one or more of the other units. For example, when R₁, R₂, are methyl and hydroxyl respectively, the resulting difunctional unit is of the formula:

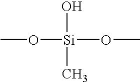

When this difunctional unit is polymerized with one or more of the other units the silicon atom will be bonded to two oxygen atoms, i.e. will share two one-halves of an oxygen atom.

The amphiphilic silicone resin emulsifier may also contain M or Q units, preferably in minor amounts which will not destroy the resinous nature of the emulsifier or compromise its amphiphilic properties.

The term “M” means a monofunctional siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units.

In silicone nomenclature used by those skilled in the art, a monofunctional siloxy unit means a unit having the general formula:
R₁R₂R₃SiO_{1/2}
wherein R₁ and R₂ are each independently as defined above, and R₃ is independently the same as R₁ and R₂. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

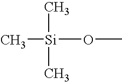

When this monofunctional unit is polymerized with one or more of the other units the oxygen atom will be shared by another silicon atom, i.e. the silicon atom in the monofunctional unit is bonded to ½ of this oxygen atom.

It is possible that the amphiphilic silicone resin emulsifier may also contain a number of Q units due to how the resin is manufactured.

The term “Q” means a “tetrafunctional siloxy unit” in standard silicone nomenclature. A “Q” unit has the general formula:
SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

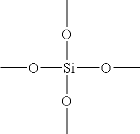

Preferred amphiphilic silicone resin emulsifiers for use in the compositions of the invention include those having the general formula:
[R₁ SiO_{3/2}]ₓ[CH₃Si(OH)O_{2/2}]_{y}
where x ranges from about 1 to 1,000,000, preferably about 1-500,000, more preferably about 1-100,000, and wherein R₁ is as defined above. Such amphiphilic silicone resin emulsifiers can be purchased from Active Concepts under the trade name SilDerm Emulsifying BG or SilDerm Emulsifying CS. SilDerm Emulsifying BG has the INCI name butylene glycol and polymethylsilsesquioxane, and is a mixture of polymethylsilsesquioxane and butylene glycol. SilDerm Emulsifying CS is a mixture of cyclopentasiloxane and polymethylsilsesquioxane.

The amphiphilic silicone resins used in the composition are made according to processes well known in the art. In general siloxane polymers are obtained by hydrolysis of silane monomers, preferably chlorosilanes, in aqueous or aqueous/alcoholic media. The chlorosilanes are hydrolyzed to silanols and then condensed to form siloxanes. For example, T units are often made by hydrolyzing tetrachlorosilanes in aqueous or aqueous/alcoholic media to form the following:

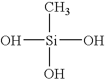

The above hydroxy substituted silane is then condensed or polymerized with other types of silanol substituted units such as: 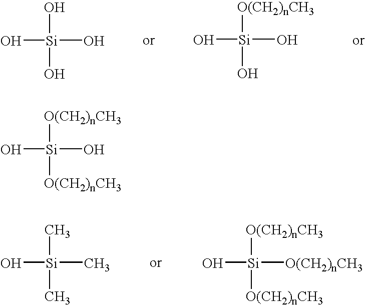
wherein n is 0-10, preferably 0-4.

Because the hydrolysis and condensation may take place in aqueous or aqueous/alcoholic media wherein the alcohols are preferably lower alkanols such as ethanol, propanol, or isopropanol, any of the units may have residual hydroxyl or alkoxy functionality as depicted above. Preferably, the resins are made by hydrolysis and condensation in aqueous/alcoholic media, which provides resins that have residual hydroxyl groups in an amount sufficient to confer the appropriate degree of hydrophilic property to the resin. In the case where the alcohol is ethanol, the result is a resin that may have residual hydroxy or ethoxy functionality on the siloxane polymer. The lipophilic portions of the amphiphilic silicone resin may be found in the siloxane backbone of the polymer. In particular, the siloxane backbone, when substituted with alkyl groups such as methyl, tends to confer lipophilicity to those portions of the molecule where they are found.

The silicone film forming polymers used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

It is noted that the amphiphilic silicone resin emulsifiers used in the compositions of the invention are distinguished from standard polymethylsilsesquioxanes that are known in the art. For example, Tospearl, a highly polymerized crosslinked polymethylsilsesquioxane is one example of an ingredient that is not suitable for use as an amphiphilic silicone resin emulsifier in the compositions of the invention because it does not have amphiphilic properties. Similarly, polysilsesquioxanes sold under the trade name Wacker MK resin are also not suitable for use as the silicone resin emulsifier in the compositions of the invention because they do not have the required amphiphilic properties. While the Wacker MK resins have some residual hydroxy and alkoxy functionality, they do not have sufficient hydroxyl functionality to perform an emulsification function.

The amphiphilic silicone resin emulsifier is used to emulsify anhydrous or emulsion cosmetic compositions having at least two internally incompatible phases. The incompatible phases may be polar and nonpolar. Other examples of incompatible phases include materials that may be generally nonpolar, for example, but have solubility parameters that are different enough so that one ingredient or combination of ingredients is not readily soluble or dispersible in the other ingredient or combination of ingredients even though they are the same type of ingredients and would generally form a combination of ingredients that would be found in the same phase of the composition (e.g. oil phase, water phase).

The term “solubility parameter” when used herein means the Hildebrand solubility parameter (6) which is calculated according to the formula:
δ=(ΔEv/V)^{1/2}
wherein ΔEv=heat of vaporization of the particular ingredient, and V=molecular weight/density of the ingredient.

The Hildebrand solubility parameters (6) are generally available by referring to standard chemistry textbooks or similar reference manuals. The Journal of the Society of Cosmetic Chemistry, Volume 36, pages 319-333, and Cosmetics and Toiletries, Vol. 103, October 1988, pages 47-69, both of which are hereby incorporated by reference in their entirety, list the Hildebrand solubility parameter (δ) values for a wide variety of cosmetic ingredients and how the solubility parameter is calculated. For example, ingredients that have a Hildebrand solubility parameter (δ) ranging from about 5 to 12 are moderately lipophilic and include ingredients such as coconut oil, mineral oil, isopropyl myristate, linseed oil, octyl palmitate, and so on. Ingredients that have solubility parameters which are significantly less than 5 tend to be more extremely lipophilic and very hydrophobic. Ingredients that have Hildebrand solubility parameters that are greater than 12 tend to be more hydrophilic in nature. In the case where the composition may contain one oily ingredient that has a Hildebrand solubility parameter of 1 or 2 and another oily ingredient that has a Hildebrand solubility parameter of 11, such ingredients may be internally incompatible in the composition due to these differences in solubility parameters, yet such ingredients would otherwise form part of the same phase in the composition.

B. Other Ingredients

The phases may contain a variety of ingredients including, but not limited to, those set forth below.

1. Water

In the case where the compositions of the invention are in the aqueous solution or water and oil emulsion form, the composition comprises from about 0.1-99%, preferably about 0.5-90%, more preferably about 1-80% by weight of the total composition of water.

2. Polar Solvents

The compositions may comprise one or more polar solvents besides water, that are generally soluble in the water to form a water phase in the case of emulsion or solution compositions. If present, such polar solvents may range from about 0.001-85%, preferably about 0.01-75%, more preferably about 0.1-65% by weight of the total composition. Suitable polar solvents include mono-, di-, or polyhydric alcohols including those having the general formula R—OH wherein R is a C₁₋₁₀ straight or branched chain alkyl that may be substituted with one or more hydroxyl groups. Also suitable are short chain (C₁₋₄) alkylene glycols. Examples of such alcohols include ethanol, isopropanol, glycerin, butylene glycol, propylene glycol, and mixtures thereof. Suitable alkylene glycols include propylene, ethylene, or butylene glycols and the like.

3. Oils

The composition preferably contains one or more oily ingredients. The term “oil” when used herein means an ingredient that is a pourable liquid at room temperature. Such oils tend to be generally nonpolar, but may contain substituents or moieties that are polar in character. The oils that may be used in the compositions of the invention are silicone oils, organic oils, or mixtures thereof. Such oils may be present ranging from about 0.1-98%, preferably 0.5-90%, more preferably about 1-80% by weight of the total composition. Such oils include, but are not limited to, those set forth herein.

(a). Silicone Oils

Silicone oils that may be used in the composition are volatile or non-volatile. The term “volatile” when used herein, means that the silicone oil has a vapor pressure of at least about 2 mm. of mercury at 20° C. The term “non-volatile” means that the oil has a vapor pressure of less than about 2 mm. of mercury at 20° C.

(i). Volatile Silicone Oils

Suitable volatile silicones include linear or cyclic volatile silicones. Suitable volatile silicone oils generally have a viscosity ranging from about 0.1 to 10, preferably about 0.1-5 centipoise at 25° C.

Cyclic silicones (or cyclomethicones) are of the general formula: 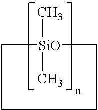
where n=3-6.

Linear volatile silicones in accordance with the invention have the general formula:
(CH₃)₃Si—O—[Si(CH₃)₂—O]ₙ—Si(CH₃)₃
where n=0, 1, 2, 3, 4, 5, 6, or 7, preferably 0-5, more preferably 1-4.

Linear and cyclic volatile silicones are available from various commercial sources including Dow Corning Corporation and General Electric. The Dow Corning volatile silicones are sold under the tradenames Dow Corning 244, 245, 344, and 200 fluids. These linear and cyclic volatile fluids include octamethylcyclotetrasiloxane, octamethyltrisiloxane, decamethyltetrasiloxane, decamethylcyclopentasiloxane, dodecamethylcyclohexasiloxane, hexamethyldisiloxane, and mixtures thereof.

(b). Hydrocarbon Oils

Also suitable are various straight or branched chain hydrocarbon oils that may be volatile or non-volatile.

(i). Volatile Hydrocarbon Oils

For example, suitable volatile hydrocarbons include straight or branched chain paraffinic hydrocarbons having 5 to 40 carbon atoms, more preferably 8-19 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight ranging from about 70-225, preferably about 160 to 190, and a boiling point range of about 30 to 320, preferably about 60 to 260° C., and a viscosity of less than about 10 centipoise at 25° C. Such paraffinic hydrocarbons are available from EXXON under the ISOPARS trademark, and from the Permethyl Corporation. Suitable C₁₂ isoparaffins having the INCI name isododecane are manufactured by Permethyl Corporation under the tradename Permethyl 99A. Various C₁₆ isoparaffins commercially available under the trade name Permethyl R and having the INCI name isohexadecane are also suitable. Another suitable type of suitable paraffin is referred to as C₉₋₁₁ isoparaffins, which is a mixture of isoparaffinic hydrocarbons having 9, 10, and 11 carbon atoms.

(ii). Non-Volatile Hydrocarbon Oils

Also suitable are various non-volatile hydrocarbon oils including isoparaffins and olefins having greater than 19 carbon atoms. Examples of such hydrocarbon oils include C₂₄₋₂₈ olefins, C₃₀₋₄₅ olefins, C₂₀₋₄₀ isoparaffins, hydrogenated polyisobutene, polybutene, mineral oil, pentahydrosqualene, squalene, squalane, and mixtures thereof. Preferably such hydrocarbons have from about 20 to 80 carbon atoms.

(c). Esters

Also suitable are various esters that may be in the form of mono-, di-, or triesters. Preferably, such esters have a viscosity ranging from about 10 to 1,000,000 centipoise at 25° C.

(i). Monoesters

Monoesters are generally formed by the reaction of a monocarboxylic acid having the formula R-COOH, wherein R is a straight or branched chain saturated or unsaturated alkyl having from 1 to 30 carbon atoms, or phenyl; and an alcohol having the formula R—OH wherein R is a straight or branched chain saturated or unsaturated alkyl having from about 1-30 carbon atoms, or phenyl. Both the alcohol and the acid may be substituted with one or more hydroxyl groups, and the carboxylic acid may be an alpha hydroxy acid. Either one or both of the acid or alcohol may be a “fatty” acid or alcohol, for example, may have from about 6 to 22 carbon atoms. Examples of monoester oils that may be used in the compositions of the invention include, but are not limited to, hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl, isostearate, butyl acetate, butyl isostearate, butyl oleate, butyl octyl oleate, cetyl palmitate, ceyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on.

(ii). Diesters

Suitable diesters that may be used in the compositions of the invention are the reaction product of a dicarboxylic acid and an aliphatic or aromatic alcohol, or a monocarboxylic acid and an aliphatic or aromatic alcohol containing at least two hydroxyl groups. The dicarboxylic acid may contain from 1 to 30 carbon atoms, and may be in the straight or branched chain, saturated or unsaturated form. The dicarboxylic acid may be substituted with one or more hydroxyl groups. The aliphatic or aromatic alcohol may also contain 1 to 30 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 6-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, isononyl isononanaote, isohexadecyl stearate, and so on.

(iii). Triesters

Suitable triesters comprise the reaction product of a tricarboxylic acid and an aliphatic or aromatic alcohol or the reaction of an aliphatic or aromatic alcohol having three or more hydroxyl groups with mono- or dicarboxylic acids. As with the mono- and diesters mentioned above, the acid and alcohol contain from 1 to 30 carbon atoms, and may be saturated or unsaturated, straight or branched chain, and may be substituted with one or more hydroxyl groups. Preferably, one or more of the acid or alcohol is a fatty acid or alcohol containing 6 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, and so on.

(d). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(e). Glyceryl Esters of Fatty Acids

Also suitable for use as the oil are various synthetic or naturally occuring glyceryl esters of fatty acids, or triglycerides. Both vegetable, animal, or synthetic sources may be used. Examples of such oils include castor oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

4. Particulates

When the compositions of the invention are colored or opaque they may contain amounts of particulates ranging from about 0.01-95%, more preferably about 0.5-18% of particulate matter having a particle size of 0.01 to 200, preferably 0.25-100 microns. The particulate matter may be colored or non-colored (for example white) non-pigmentitious powders that may give the composition an opaque or semi-opaque quality. Suitable non-pigmentitious powders include bismuth oxychloride, titanated mica, fumed silica, spherical silica, polymethylmethacrylate, micronized teflon, boron nitride, acrylate copolymers, aluminum silicate, aluminum starch octenylsuccinate, calcium silicate, cellulose, chalk, corn starch, glyceryl starch, hydrated silica, kaolin, maltodextrin, microcrystalline cellulose, rice starch, silica, talc, mica, titanium dioxide, zinc laurate, zinc myristate, zinc rosinate, alumina, calcium carbonate, dextran, nylon, silica silylate, silk powder, sericite, soy flour, tin oxide, titanium hydroxide, trimagnesium phosphate, walnut shell powder, or mixtures thereof. While titanium dioxide is commonly considered to be a white pigment when used in paints, in cosmetics it is used more for its ability to mute color, and/or provide an opaque or semi-opaque finish, then as a colorizing ingredient. The above mentioned powders may be surface treated with lecithin, amino acids, mineral oil, silicone, or various other agents either alone or in combination, which coat the powder surface and render the particles more lipophilic in nature. In some cases the particulates may be in the form of fibers, which have a cross-sectional shape and some degree of length which may range from 0.1 mm. or greater. Examples of such fibers include silk, nylon, cellulose, rayon, teflon, and other types of synthetic or natural materials.

The particulate matter component also may comprise various organic and/or inorganic pigments, alone or in combination with one or more non-pigmentatious powders.

The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The composition may contain a mixture of both pigmentatious and non-pigmentatious particulate matter. The percentage of pigment used in the particulate matter component will depend on the type of cosmetic being formulated. Preferred is where the particulate phase comprises a mixture of pigmentitious and non-pigmentitious particulate matter, generally ranging from about 0.1-80% pigmentitious particulate matter to about 0.1-90% non-pigmentitious particulate.

5. Thickening Agents

It may be desirable to include one or more thickening agents in the compositions. Thickening may be achieved by waxes or monmorillonite minerals, or various types of associative thickeners. If present, suggested ranges of thickening agent are from about 0.01-75%, preferably about 0.1-65%, more preferably about 0.5-50% by weight of the total composition.

Suitable waxes include animal, vegetable, mineral, and synthetic waxes, or silicone waxes. Generally such waxes have a melting point ranging from about 28 to 125° C., preferably about 30 to 100° C. Examples of waxes include acacia, beeswax, ceresin, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG 6-20 beeswax, or PEG-12 carnauba wax.

Also suitable are various types of silicone waxes, referred to as alkyl silicones, which are polymers that comprise repeating dimethylsiloxy units in combination with one or more methyl-long chain alkyl siloxy units wherein the long chain alkyl is generally, a fatty chain that provides a wax-like characteristic to the silicone. Such silicones include, but are not limited to stearoxydimethicone, behenoxy dimethicone, stearyl dimethicone, cetearyl dimethicone, and so on.

Suitable waxes are also set forth in U.S. Pat. No. 5,725,845 which is hereby incorporated by reference in its entirety.

If present, suitable montmorillonite minerals include natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, and bentones, either alone or in combination with carbonate activators such as propylene carbonate.

Other types of thickening agents include fatty acids or alcohols, optionally substituted with hydroxyl groups, for example 12-hydroxystearic acid. Such fatty acids or alcohols have the general formula R—COOH or R—OH respectively, where R is a straight or branched chain, saturated or unsaturated alkyl having from about 6 to 45 carbon atoms, wherein one or more of the alkyl groups may be substituted with functional groups such as hydroxy, alkoxy, alkyl, and so on.

6. Emulsifiers

The compositions of the invention may be emulsified with only the amphiphilic silicone resin emulsifier described herein, or if desired, one or more additional organic or silicone surfactants may be present as co-emulsifiers. In one embodiment of the invention, the composition is free of emulsifiers other than the amphiphilic silicone resin emulsifier. In another embodiment of the invention, the composition contains at least one additional organic or silicone co-emulsifier. If present, the co-emulsifer may range from about 0.001-40%, preferably about 0.1-15%, more preferably about 0.5-10% by weight of the total composition of at least one co-emulsifier. The emulsifiers present may be in the nonionic, cationic, anionic, zwitterionic, or amphoteric form. Preferably, if co-emulsifiers are present they are nonionic.

(a). Nonionic Organic Emulsifiers

Suitable nonionic surfactants or emulsifiers include alkoxylated alcohols, or ethers, formed by the reaction of an alcohol with an alkylene oxide, usually ethylene or propylene oxide. Preferably the alcohol is a fatty alcohol having 6 to 30 carbon atoms. Examples of such ingredients include Beheneth 5-30, which is formed by the reaction of behenyl alcohol and ethylene oxide where the number of repeated ethylene oxide units ranges from 5 to 30; Ceteareth 2-100, formed by the reaction of a mixture of cetyl and stearyl alcohol with ethylene oxide, where the number of repeating ethylene oxide units in the molecule ranges from 2 to 100; Ceteth 1-45 which is formed by the reaction of cetyl alcohol and ethylene oxide, and the number of repeating ethylene oxide units ranges from 1 to 45, and so on. Other alkoxylated alcohols are formed by the reaction of fatty acids, mono-, di- or polyhydric alcohols, and alkylene oxides. For example, compounds formed by the reaction of C₆₋₃₀ fatty carboxylic acids, polyhydric alcohols (such as monosaccharides such as glucose, galactose, glycerin, methyl glucose) and an alkoxylated alcohol (such as steareth, beheneth, ceteareth, and the like) are also suitable.

Also suitable as the nonionic surfactant are alkyoxylated carboxylic acids, which are formed by the reaction of a carboxylic acid with an alkylene oxide or with a polymeric ether. The resulting products have the general formula: 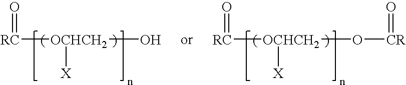
where R is a C₁₋₃₀ straight or branched chain saturated or unsaturated alkyl, X is hydrogen or lower alkyl, and n is the number of polymerized alkoxy groups, which may range from 2 to 100,000. In the case of the diesters, the two RCO— groups do not need to be identical.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula: 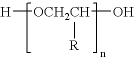
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Also, particularly suitable as nonionic organic emulsifiers are various types of esters of fatty acids and glycerin or polyglycerin. Examples of such fatty acid esters include glyceryl stearate, diglyceryl diiosostearate, polyglyceryl-3-isostearate, polyglyceryl-4 isostearate, polyglyceryl-6 ricinoleate, polyglyceryl-4-diisostearate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(b). Silicone Surfactants

Suitable silicone co-emulsifiers include those that have a polymeric backbone having siloxy units that have linear repeating units, e.g. di(lower)alkylsiloxy units, preferably dimethylsiloxy units. The silicone co-emulsifier has a hydrophilic portion, which is generally achieved by substitution onto the polymeric backbone of a radical that confers hydrophilic properties to a portion of the molecule. The hydrophilic radical may be substituted on a terminus of the silicone, or on any one or more repeating units of the polymer. In general, the repeating dimethylsiloxy units of modified polydimethylsiloxane emulsifiers are lipophilic in nature due to the methyl groups, which confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the silicone co-emulsifier, if used in the compositions of the invention, should have at least one hydrophilic portion and one lipophilic portion.

The term “hydrophilic radical” means a radical that, when substituted onto the silicone polymer backbone, confers hydrophilic properties to the substituted portion of the polymer. Examples of radicals that will confer hydrophilicity are hydroxy-polyethyleneoxy, hydroxyl, carboxylates, sulfonates, sulfates, phosphates, or amines.

The term “lipophilic radical” means an organic radical that, when substituted onto the silicne polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. The C₁₋₄₀ alkyl may be non-interrupted, or interruped by one or more oxygen atoms, a benzene ring, amides, esters, or other functional groups.

The silicone co-emulsifiers may have the following general formula: 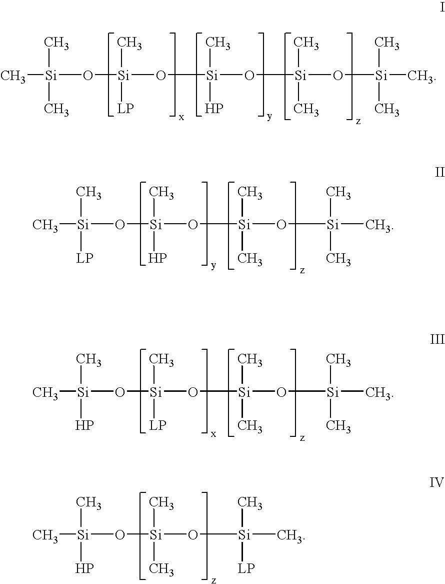
wherein LP is a lipophilic radical
HP is a hydrophilic radical
x is 0-5000
y is 0-5000, and
z is 0-5000, with the proviso that the organosiloxane contains at least on hydrophilic radical and at least one lipophilic radical.

More preferred are compounds of the generic formula I, above, wherein LP is a lipophilic radical which is a C₁₋₄₀ straight or branched chain alkyl, HP is a hydrophilic radical containing hydroxy-polyethyleneoxy. Most preferred is a compound of the formula: 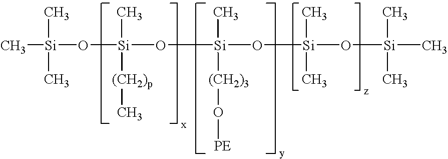
wherein p is 10-40, preferably 12-20, most preferably 15, a is 1-50,000, b is 1-50,000, and
PE is (—C₂H₄O)ₐ(—C₃H₆O)_{b}—H
where x, y, z, a, and b are such that the maximum molecular weight of the polymer is approximately 50,000. Silicone emulsifiers useful in the compositions of the invention are commercially available from Goldschmidt Corporation under the ABIL tradename. One type of such emulsifier is cetyl dimethicone copolyol and has the tradename ABIL WE 09 or ABIL WS 08. The cetyl dimethicone copolyol may be used alone or in conjunction with other non-silicone organic emulsifiers. For example, the cetyl dimethicone copolyol may be in a mixture with other non-silicone organic emulsifiers and emollients. In particular, blends of 25-50% of the organosiloxane emulsifier, 25-50% of a non-silicone organic emulsifier, and 25-50% by weight emollients or oils are preferred. For example, the mixtures identified by the C.T.F.A. names cetyl dimethicone copolyol (and) polyglyceryl 4-isostearate (and) hexyl laurate, or cetyl dimethicone copolyol (and) polyglyceryl-3 oleate (and) hexyl laurate both work well. These blends contain approximately 25-50% of each ingredient, for example ABIL WE 09 contains approximately, by weight of the total ABIL composition, 25-50% cetyl dimethicone copolyol, 25-50%, polyglyceryl 4-isostearate, and 25-50% of hexyl laurate which is an emollient or oil.

Another type of silicone co-emulsifier suitable for use in the compositions of the invention are emulsifiers sold by Union Carbide under the Silwet™ trademark. These emulsifiers are represented by the following generic formulas:
(Me₃Si)_{y-2}[(OSiMe₂)_{x/y}O—PE]_{y}
wherein
PE=—(EO)ₘ(PO)ₙR
R=lower alkyl or hydrogen
Me=methyl
EO is polyethyleneoxy
PO is polypropyleneoxy
m and n are each independently 1-5000
x and y are each independently 0-5000, and 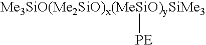
   wherein
PE=—CH₂CH₂CH₂O(EO)ₘ(PO)ₙZ
Z=lower alkyl or hydrogen, and
Me, m, n, x, y, EO and PO are as described above,
   with the proviso that the molecule contains a lipophilic portion and a hydrophilic portion. Again, the lipophilic portion can be supplied by a sufficient number of methyl groups on the polymer backbone.

One particular type of silicone co-emulsifier is sold under the Silwet™ brand and has the following general formula: 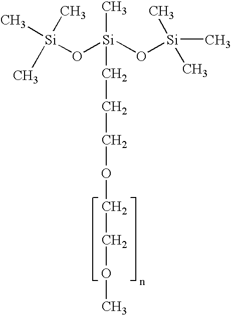
Wherein n is 1-10, preferably 8.

Generally silicone co-emsulfiers suitable for use in the compositions of the invention are known by the INCI name dimethicone copolyol and cetyl dimethicone copolyol.

Examples of other silicone emulsifiers include amino/polyoxyalkyleneated polydiorganosiloxanes disclosed in U.S. Pat. No. 5,147,578. Also suitable are organosiloxanes sold by Goldschmidt under the ABIL trademark including ABIL B-9806, as well as those sold by Rhone-Poulenc under the Alkasil tradename. Also, organosiloxane emulsifiers sold by Amerchol under the Amersil tradename, including Amersil ME-358, Amersil DMC-287 and Amersil DMC-357 are suitable. Dow Corning surfactants such as Dow Corning 3225C Formulation Aid, Dow Corning 190 Surfactant, Dow Corning 193 Surfactant, Dow Corning Q2-5200, and the like are also suitable

Suitable cationic, anionic, zwitterionic, and amphoteric surfactants are disclosed in U.S. Pat. No. 5,534,265, which is hereby incorporated by reference in its entirety.

6. Sunscreens

If desired, the compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmits UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on pages 1808-1809 of the CTFA Cosmetic Ingredient Dictionarv and Handbook, Eighth Edition, 2000, as well as U.S. Pat. No. 5,620,965, both of which are hereby incorporated by reference. Further examples of chemical and physical sunscreens include those set forth below.

(a). UVA Chemical Sunscreens

The term “UVA sunscreen” means a chemical compound that blocks UV radiation in the wavelength range of about 320 to 400 nm. Preferred UVA sunscreens are dibenzoylmethane compounds having the general formula: 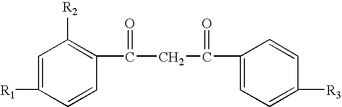
wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

If present the sunscreens may be found ranging from about 0.001-20%, preferably 0.005-5%, more preferably about 0.005-3% by weight of the composition of UVA sunscreen.

(b). UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. Particularly preferred is Octocrylene, which is 2-ethylhexyl 2-cyano-3,3-diphenylacrylate. Preferred is where the composition contains no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula: 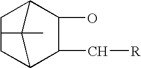
wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula: 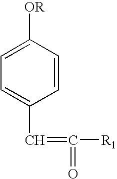
wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at nor more than about 3% by weight of the total composition.

Also suitable as the UVB screening agents are various benzophenone derivatives having the general formula: 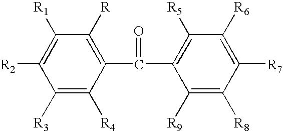
R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula: 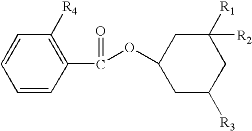
wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

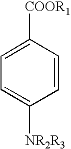

Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: 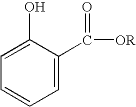
wherein R is a straight or branched chain alkyl, including derivatives of the above compound formed from mono-, di-, or triethanolamines. Particular preferred are octyl salicylate, TEA-salicylcate, DEA-salicylate, and mixtures thereof.

Generally, the amount of the UVB chemical sunscreen present may range from about 0.001-45%, preferably 0.005-40%, more preferably about 0.01-35% by weight of the total composition.

(c). Physical Sunscreens

The composition may also contain one or more physical sunscreens. The term “physical sunscreen” means a material that is generally particulate in form that is able to block UV rays by forming an actual physical block on the skin. Examples of particulates that serve as solid physical sunblocks include titanium dioxide, zinc oxide and the like in particle sizes ranging from about 0.001-50 microns, preferably less than 1 micron.

7. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

8. Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, famesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.

II. The Method

The invention further comprises a method for emulsifying an anhydrous or emulsion cosmetic composition containing at least two internally incompatible phases, with an amphiphilic silicone resin emulsifier either alone or in combination with a secondary emulsifier. The silicone resin emulsifier may be used alone as the emulsifier in the compositions of the invention, or, if desired, may be used in combination with other organic or silicone-based emulsifiers including those described herein. The silicone resin emulsifier is used in the amounts set forth herein, and will facilitate compatibility between at least two different phases or ingredients in the composition to form an internally compatible cosmetic composition.

The invention will be further described in connection with the following examples, which are set forth for the purposes of illustration only.

### EXAMPLE 1

A water in oil emulsion foundation makeup composition was prepared as follows:

| | | % by |
|---|---|---|
| Seq. | Ingredient | weight |
| 1 | Cyclomethicone | 5.00 |
| 1 | Propyl paraben | 0.10 |
| 1 | Titanium dioxide, cyclomethicone, dimethicone copolyol, | 13.50 |
| | polyglyceryl-6-ricinoleate, stearic acid, aluminum | |
| | hydroxide | |
| 1 | Zinc oxide, cyclomethicone, dimethicone copolyol, | 3.50 |
| | dimethicone | |
| 1 | Titanium dioxide, cyclomethicone, dimethicone copolyol, | 4.85 |
| | triethoxycaprylyl silane | |
| 1 | Iron oxides, methicone | 0.96 |
| 1 | Iron oxides, methicone, boron nitride | 0.64 |
| 1 | Iron oxides, methicone | 0.12 |
| 1 | Mica, methicone | 1.60 |
| 1 | Boron nitride | 0.82 |
| 1 | Silica | 0.10 |
| 1 | Nylon-12 | 1.00 |
| 1 | Titanium dioxide, iron oxides | 0.15 |
| 1 | Titanium dioxide, iron oxides | 0.15 |
| 1 | Titanium dioxide, alumina, methicone | 0.75 |
| 2 | Polyglyceryl-4-isostearate, cetyl dimethicone copolyol, | 3.75 |
| | hexyl laurate | |
| 2 | Cyclomethicone, dimethicone | 3.90 |
| 2 | Cyclomethicone, trimethylsiloxysilicate | 2.50 |
| 2 | Dimethicone | 6.70 |
| 2 | Cyclomethicone | 4.04 |
| 2 | Isostearic acid | 0.45 |
| 3 | Tribehenin | 0.10 |
| 4 | Water | QS |
| 4 | Magnesium sulfate | 1.00 |
| 4 | Tetrasodium EDTA | 0.01 |
| 4 | Triethanolamine | 0.15 |
| 4 | Aloe barbadensis leaf juice | 0.10 |
| 4 | Sodium hyaluronate, hydrolyzed glycosaminoglycans | 0.30 |
| 4 | Saccharide isomerate | 0.50 |
| 5 | Glycerin | 2.00 |
| 5 | Butylene glycol | 4.00 |
| 5 | Polymethylsilsesquioxane and butylene glycol | 5.00 |
| 5 | Methyl paraben | 0.25 |
| 6 | Kinetin | 0.05 |
| 6 | Sodium hydroxide | 0.05 |
| 7 | Dimethicone, cyclomethicone | 2.80 |
| 8 | Cyclomethicone, dimethiconol | 2.00 |
| 8 | Tocopheryl acetate | 0.01 |
| 8 | Retinyl palmitate | 0.01 |
| 8 | Cyclomethicone | 1.00 |
| 8 | Cyclomethicone, gingko biloba extract, panax ginseng | 0.50 |
| | root extract, Camilla sinensis leaf extract, c entaurea | |
| | cyanus flower extract, vitis vinifera extract | |
| 8 | Anthemis Nobilis flower oil | 0.20 |

The composition was made by combining the materials in sequence 1 to form a grind. The sequence 2 and 3 ingredients were combined and mixed with the sequence 1 ingredients. Separately, the sequence 4, 5, and 6 ingredients were combined and emulsified into the mixture of sequences 1, 2, and 3. The sequence 7 and 8 ingredients were combined and mixed with the mixture to form a foundation makeup.

### EXAMPLE 2

A waterproof mascara formula was made as follows:

| | | % by |
|---|---|---|
| Seq. | Ingredient | weight |
| 1 | Butylene glycol and polymethylsilsesquioxane¹ | 4.50 |
| 2 | Cyclomethicone, disteardimonium hectorite, propylene | 5.50 |
| | carbonate | |
| 3 | Tocopheryl acetate | 0.10 |
| 4 | Carnauba wax | 6.00 |
| 5 | Paraffin | 9.00 |
| 6 | Sorbic acid | 0.20 |
| 7 | HDI/trimethylol hexyllactone crosspolymer, silica | 1.00 |
| 8 | Polyethylene | 3.00 |
| 9 | Isododecane | 23.33 |
| 10 | Trimethylsiloxysilicate | 5.00 |
| 11 | Iron oxides | 13.00 |
| 12 | Phenoxyethanol | 1.00 |
| 13 | Propyl paraben | 0.15 |
| 14 | Trihydroxystearin | 2.50 |
| 15 | Dimethicone crosspolymer-3, isododecane | 3.00 |
| 16 | Water | QS |
| 17 | Simethicone | 0.35 |
| 18 | Magnesium ascorbyl phosphate | 0.01 |
| 19 | Methyl paraben | 0.35 |
| 20 | Butylene glycol | 1.00 |
| 21 | Retinyl palmitate | 0.01 |

The composition was made by combining the ingredients of each sequence and mixing well to form a mascara composition.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. An internally compatible cosmetic composition comprising at least two internally incompatible phases that are compatibilized with an effective amount of an amphiphilic silicone resin emulsifier.

2. The composition of claim 1 wherein the composition is free of emulsifiers other than the amphiphilic silicone resin emulsifier.

3. The composition of claim 1 wherein the amphiphilic silicone resin emulsifier is present with at least one additional silicone or organic co-emulsifier.

4. The composition of claim 1 wherein the amphiphilic silicone resin emulsifier is a silicone comprised of T units, either alone or in combination with D units, wherein the T unit has the general formula:
R₁ SiO_{3/2}
and the D unit has the general formula:
R₁R₂SiO_{2/2}
wherein R₁ and R₂ are each independently a C₁₋₃₀ straight or branched chain alkyl, which may be substituted with phenyl or one or more hydroxyl groups; hydroxyl; phenyl; C₁₋₂₂ alkoxy; or hydrogen.

5. The composition of claim 4 wherein the D units present contain hydroxyl substituents.

6. The composition of claim 4 wherein the T unit, R₁ is C₁₋₂₂ straight or branched chain alkyl.

7. The composition of claim 6 wherein the T unit, R₁ is a C₁₋₁₀ alkyl.

8. The composition of claim 7 wherein the T unit, R₁ is methyl.

9. The composition of claim 4 wherein the D unit R₁ is a C₁₋₂₂ straight or branched chain alkyl.

10. The composition of claim 9 wherein the D unit R₂ is hydroxyl.

11. The composition of claim 10 wherein the D unit R₁ is methyl.

12. The composition of claim 1 wherein the amphiphilic silicone resin emulsifier comprises T units and D units where the T unit has the general formula:
R₁ SiO_{3/2}
and the D unit has the general formula:
R₁R₂SiO_{2/2}
wherein R₁ is methyl and R₂ is hydroxyl.

13. The composition of claim 12 wherein the amphiphilic silicone resin emulsifier has the general formula:
[CH₃SiO_{3/2}]ₓ[CH₃Si(OH)O_{2/2}]_{y}
wherein x and y are each independently 1 to 1,000,000.

14. The composition of claim 1 additionally comprising a secondary emulsifier.

15. The composition of claim 1 wherein the secondary emulsifier is a silicone emulsifier.

16. The composition of claim 1 wherein the silicone emulsifier is selected from the group consisting of: 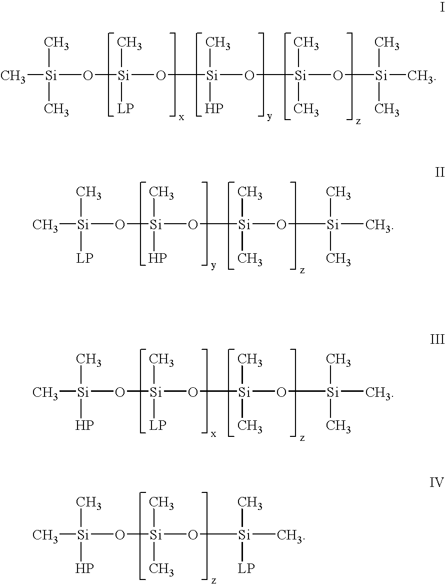
wherein LP is a lipophilic radical
HP is a hydrophilic radical
x is 0-5000
y is 0-5000, and
z is 0-5000, with the proviso that the silicone contains at least on hydrophilic radical and at least one lipophilic radical.

17. The composition of claim 16 wherein the silicone co-emulsifier is of Formula I, above, wherein LP is C₁₀₄₀ and HP is PE where PE is (—C₂H₄O)ₐ(C₃H₆O)_{b}H, wherein a is 1-50,000 and b is 1-50,000.

18. The composition of claim 15 wherein the silicone co-emulsifier comprises cetyl dimethicone copolyol.

19. The composition of claim 16 wherein the silicone co-emulsifier is of Formula II wherein HP is PE, wherein PE is (—C₂H₄O)ₐ(C₃H₆O)_{b}Hj.

20. The composition of claim 15 wherein the silicone co-emulsifier comprises dimethicone copolyol.

21. The composition of claim 15 wherein the silicone co-emulsifier comprises a mixture of dimethicone copolyol and cetyl dimethicone copolyol.

22. The composition of claim 14 wherein the co-emulsifier comprises an organic emulsifier.

23. The composition of claim 22 wherein the organic emulsifier comprises a nonionic organic emulsifier which is an alkoxylated alcohol, alkoxylated carboxylic acid, homo- or copolymeric ether, alkoxylated sorbitan, or fatty acid esters of polyglycerin.

24. The composition of claim 14 wherein the organic emulsifier comprises esters of fatty acids and polyglycerin.

25. The composition of claim 14 wherein the organic co-emulsifier comprises diglyceryl diisostearate, polyglyceryl-3-isostearate, polyglycery-4-isostearate, polyglyceryl-6-ricinoleate, polyglyceryl-4-diisostearate, or mixtures thereof.

26. The composition of claim 1 further comprising one or more co-emulsifiers that are dimethicone copolyol, cetyl dimethicone copolyol, or esters of fatty acids and polyglycerin.

27. The composition of claim 1 further comprising a mixture of silicone and organic co-emulsifiers wherein the silicone emulsifiers are selected from the group consisting of cetyl dimethicone copolyol, dimethicone copolyol and mixtures thereof, and the organic emulsifier is one or more fatty acid esters of polyglycerin.

28. The composition of claim 27 wherein the fatty acid ester of polyglycerin is selected from the group consisting of polyglyceryl-6-ricinoleate, polyglyceryl-3-isostearate, polyglyceryl-4-isostearate, polyglyceryl-4-diisostearate, diglyceryl diisostearate, diglyceryl isostearate, and mixtures thereof.

29. The composition of claim 1 which is in the emulsion form.

30. The composition of claim 29 wherein the internally incompatible phases are water and oil phases.

31. The composition of claim 30 comprising from about 0.001-99% water and from about 0.1-98% oil.

32. The composition of claim 31 wherein one or more of the oils are silicone oils.

33. The composition of claim 32 wherein the silicone oils are volatile silicones, non-volatile silicones, or mixtures thereof.

34. The composition of claim 33 wherein the volatile silicones are linear or cyclic silicones selected from the group consisting of decamethylcyclopentasiloxane, dodecamethylcyclohexasiloxane, octamethyltrisiloxane, decamethyltetrasiloxane, dodecamethylpentasiloxane, and mixtures thereof.

35. The composition of claim 33 wherein the non-volatile silicones are dimethicone, phenyl trimethicone, phenyl dimethicone, or mixtures thereof.

36. The composition of claim 31 which is a foundation makeup composition comprising, by weight of the total composition:
about 1-80% water,
about 0.005-75% of the amphiphilic silicone resin emulsifier,
about 1-80% silicone oil comprising a mixture of volatile and non-volatile silicone oils;
about 0.01-95% particulates.

37. The composition of claim 36 wherein the amphiphilic silicone resin emulsifier has the general formula:
[CH₃SiO_{3/2}]ₓ[CH₃Si(OH)O_{2/2}]_{y}
wherein x and y are each independently 1 to 1,000,000.

38. The composition of claim 37 wherein the amphiphilic silicone resin emulsifier comprises polymethylsilsesquioxane.

39. The composition of claim 37 wherein the volatile silicone oil comprises decamethylcyclopentasiloxane.

40. The composition of claim 37 wherein the non-volatile silicone comprises dimethicone.

41. The composition of claim 37 further comprising a co-emulsifier which is a silicone emulsifier, an organic emulsifier, or mixtures thereof.

42. The composition of claim 41 wherein the co-emulsifier is a mixture of silicone and organic emulsifiers.

43. The composition of claim 42 wherein the silicone emulsifier is dimethicone copolyol, cetyl dimethicone copolyol, or mixtures thereof; and the organic emulsifier is a fatty acid ester of polyglycerin.

44. The composition of claim 43 wherein the fatty acid of polyglycerin is selected from the group consisting of polyglyceryl-3-isostearate, polyglyceryl-3-diisostearate, polyglyceryl-4-isostearate, polyglyceryl-4-diisostearate, polyglyceryl-6-ricinoleate, and mixtures thereof.

45. The composition of claim 1 which is an anhydrous color cosmetic composition comprising from about 0.1-98% of on or more oils and from about 0.01-95% particulates.

46. The composition of claim 45 wherein the oils include one or more silicone oils.

47. The composition of claim 45 wherein the particulates are a mixture of pigments and non-pigmentitious powders.

48. A water-in-oil emulsion foundation makeup composition comprising water, silicone oil, pigments, and a silicone surfactant, wherein the water and oil phases are emulsified with an amphiphilic silicone resin emulsifier having the general formula:
[CH₃SiO_{3/2}]ₓ[CH₃Si(OH)O_{2/2}]_{y}
wherein x and y are each independently 1 to 1,000,000.

49. The composition of claim 48 additionally comprising an organic surfactant that is a fatty acid ester of polyglycerin.

50. The composition of claim 49 wherein the composition further comprises one or more of a polar solvent, botanical ingredient, antioxidant, gellant, sunscreen, or mixtures thereof.
